(19) United States
(12) Patent Application Publication
(10) Pub. No.: US 2020/0288959 A1
(43) Pub. Date: Sep. 17, 2020

(54) INTRAORAL SCANNER AND TIP ASSEMBLY

(71) Applicant: PaloDEx Group OY, Tuusula (FI)

(72) Inventors: Antti Lahti, Espoo (FI); Timo Klemola, Lahela (FI); Antti Korpela, Kerava (FI); Jorma Savolainen, Helsinki (FI); Petri Hyypiä, Helsinki (FI)

(21) Appl. No.: 16/662,436

(22) Filed: Oct. 24, 2019

Related U.S. Application Data
(60) Provisional application No. 62/816,515, filed on Mar. 11, 2019.

(51) Int. Cl. A61B 1/253; A61B 1/247

(52) U.S. Cl. CPC A61B 1/253; A61B 1/247

(57) ABSTRACT

A tip assembly for an intraoral scanner includes a tip support body having a first end, a second, opposite end, and an outer surface located between the first end and the second end. The outer surface defines at least one protrusion or recess. The tip assembly further includes a disposable, flexible mirror strip having a first end, a second, opposite end, and a mirror fixed to the second end. The flexible mirror strip defines at least one protrusion or recess that engages the at least one protrusion or recess of the tip support body to releasably couple the flexible mirror strip to the tip support body. The tip assembly further includes a tip sleeve having a first end and a second, opposite end. The tip sleeve is sized and shaped to slide over at least a portion of both the tip support body and the flexible mirror strip.

### CROSS REFERENCE TO RELATED APPLICATIONS

This application claims priority to U.S. Patent Application No. 62/816,515, filed Mar. 11, 2019, the contents of which are incorporated in their entirety by reference.

### FIELD OF THE INVENTION

Embodiments relate to intraoral scanners, and more particularly to tip assemblies for intraoral scanners.

### SUMMARY OF THE INVENTION

Intraoral scanners are commonly used to scan and record anatomical geometry within a patient's mouth, and include an optical element disposed at a tip of the intra-oral scanner.

In one aspect, embodiments provide a tip assembly for an intraoral scanner, the tip assembly including a tip support body having a first end, a second, opposite end, and an outer surface located between the first end and the second end. The outer surface defines at least one protrusion or recess. The tip assembly further includes a disposable, flexible mirror strip having a first end, a second, opposite end, and a mirror fixed to the second end. The flexible mirror strip may define at least one protrusion or recess that engages the at least one protrusion or recess of the tip support body to releasably couple the flexible mirror strip to the tip support body. The tip assembly further includes a tip sleeve having a first end and a second, opposite end. The tip sleeve is sized and shaped to slide over at least a portion of both the tip support body and the flexible mirror strip. Because the flexible mirror strip is disposable, mirror quality is not compromised by cleaning methods, for example, autoclaving, that are used for existing, permanently-attached mirrors. Instead, a new disposable mirror may be attached after use, helping to ensure consistent and high mirror quality for each use of the intraoral scanner.

In another aspect, embodiments provide an intraoral scanner. The intraoral scanner includes a tip assembly, an electrical current source, and an electronic processor configured to determine a tip type of the tip assembly.

Other features and aspects of will become apparent by consideration of the following detailed description and accompanying drawings.

### BRIEF DESCRIPTION OF THE DRAWINGS

FIG. 1 is a perspective view of a tip assembly according to one embodiment for an intraoral scanner, illustrating a tip support body, a flexible mirror strip, and a tip sleeve.

FIG. 2 is an exploded view of the tip assembly.

FIG. 3 is a perspective view of the flexible mirror strip.

FIG. 4 is a perspective view of a set of multiple tip support bodies, multiple flexible mirror strips, and multiple tip sleeves.

FIG. 5 is a perspective view of a set of fully assembled tip assemblies.

FIGS. 6A-6C are perspective views of a tip assembly according to another embodiment for an intraoral scanner, illustrating an assembly process.

FIGS. 7A and 7B are perspective view of a tip assembly according to another embodiment for an intraoral scanner, illustrating an assembly process.

FIG. 8 is a perspective view of a tip assembly according to another embodiment for an intraoral scanner, illustrating an assembly process.

FIGS. 9 and 10 are perspective views of a tip assembly according to another embodiment for an intraoral scanner, illustrating an assembly process.

FIG. 11 is a perspective view of a tip assembly according to another embodiment for an intraoral scanner, illustrating an assembly process.

FIGS. 12-15 are perspective view of a tip assembly according to another embodiment for an intraoral scanner, illustrating an assembly process.

FIG. 16 illustrates an intraoral scanning system according to one embodiment.

FIG. 17 illustrates an example of an electrical contact being used to identify a tip type according to one embodiment.

Before any embodiments are explained in detail, it is to be understood that they are not limited in their application to the details of construction and the arrangement of components set forth in the following description or illustrated in the following drawings. Other embodiments are possible and embodiments are capable of being practiced or of being carried out in various ways. Also, it is to be understood that the phraseology and terminology used herein is for the purpose of description and should not be regarded as limiting.

### DETAILED DESCRIPTION

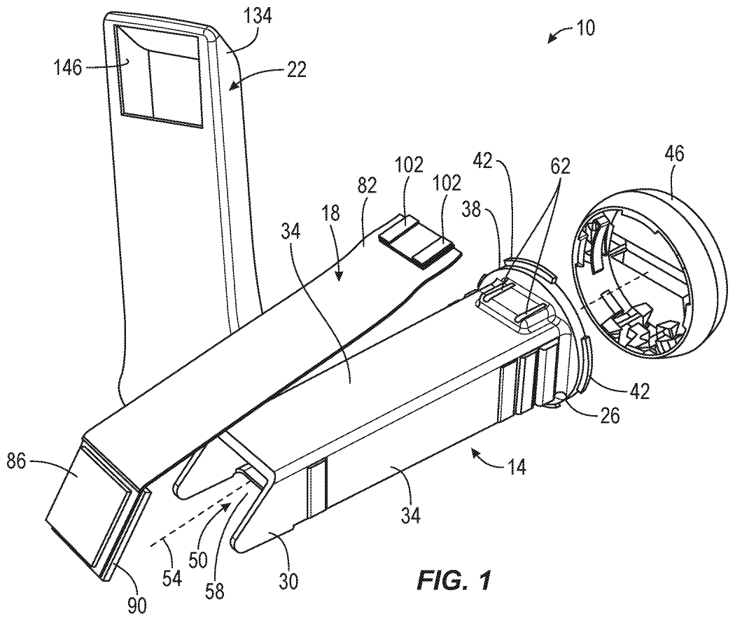

A tip assembly 10 for an intraoral scanner is shown in FIGS. 1-5. The tip assembly 10 includes a tip support body 14, a flexible mirror strip 18, and a tip sleeve 22. The tip assembly 10 may be used in combination with an intraoral scanner. The tip support body 14 includes a first end 26, a second, opposite end 30, and at least one outer surface 34 extending between the first end 26 and the second end 30. In the illustrated embodiment the tip support body 14 may be autoclaved and/or sterilized for repeated use. For example, the tip support body 14 may be subjected to UV light, or liquid disinfectant, to sterilize the tip support body 14. In other embodiments the tip support body 14 may be disposable. As illustrated in FIG. 1, the tip support body 14 may be generally rectangular in shape along a substantial portion of the tip support body 14, although in other embodiments the tip support body 14 may have a cylindrical shape or other shape not illustrated.

With reference to FIGS. 1 and 2, the first end 26 may include an outer (e.g., cylindrical) flange 38 and a series of ribs or protrusions 42 extending from the flange 38 that may be used to releasably couple and lock (e.g., via a bayonet connection) the tip support body 14 to another element 46 (e.g., adapter, housing, or other main body or structure of the intraoral scanner). Other embodiments include other shapes or sizes for the flange 38 and protrusions 42, and/or include other structures for releasably coupling the tip support body 14 to the element 46 or otherwise to the intraoral scanner. While not illustrated, the intraoral scanner may include a device (e.g., a 2D camera or 3D scanner) that receives images from the tip assembly 10 and uses those images to record topographic characteristics of a patient's set of teeth.

With continued reference to FIGS. 1 and 2, in the illustrated embodiment a cavity 50 extends axially through a length of the tip support body 14 from the first end 26 to the second end 30 along an axis 54, such that the tip support body 14 is hollow on the inside, and such that light may pass through the interior of the tip support body 14 generally along the cavity 50 and the axis 54. In the illustrated embodiment, the cavity 50 is generally rectangular in cross-section, although in other embodiments the cavity 50 may be circular in cross-section, or have other shapes than that illustrated.

The second end 30 of the tip support body 14 may be angled or tapered relative to the axis 54 (e.g., at a 45 degree angle or other angle). For example, the second end 30 may include two tapered arms separated by a gap. A cutout region or regions 58 may be provided along one or more sides of the tip support body 14 at the tapered second end 30 to allow light within a patient's mouth to enter the tip support body 14 at a defined angle, where the light is then redirected and passed through the cavity 50 to the remainder of the intraoral scanner (e.g., to a camera). The light may be redirected by a reflective surface which may be, for example, a mirror as described below.

With continued reference to FIGS. 1 and 2, the tip assembly 10 also includes a heating system to heat an optical element in the tip assembly 10 and prevent condensation build-up. For example, in the illustrated embodiment the tip support body 14 includes a set of first electrical contacts 62 that extend partially through the flange 38 and are generally positioned along and exposed alongside one of the outer surfaces 34. As described further herein, the first electrical contacts 62 provide electrical current to a resistor to heat a mirror inside the tip assembly 10 and remove condensation build-up on the mirror.

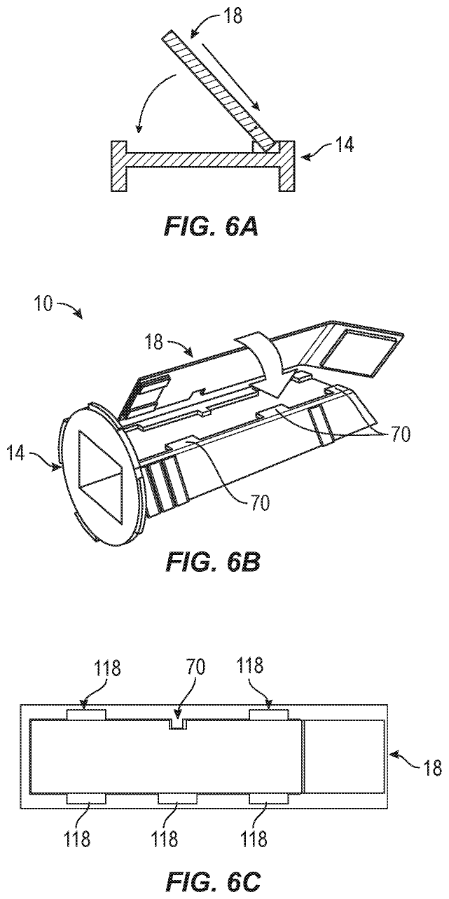

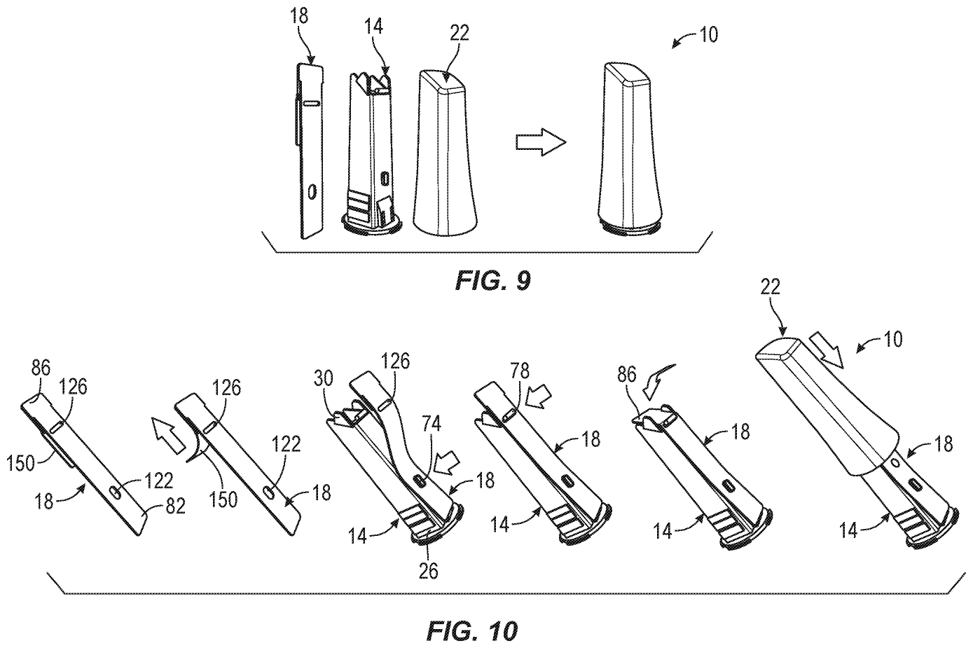

With reference to FIGS. 1-5, the tip support body 14 is releasably coupled to the flexible mirror strip 18. For example, the outer surface(s) 34 of the tip support body 14 defines at least one protrusion (e.g., raised area, projection, protruding ledge, sliding stop, knob, bump, rib, tab, rail, etc.) or recess (e.g., aperture, indentation, groove, channel, etc.) that is used to releasably and mechanically couple the tip support body 14 to the flexible mirror strip 18 via a frictional engagement (e.g., snap-on), bayonet connection, and/or other mechanical connection. For example, as illustrated in FIG. 2, the tip support body 14 may include one or more recesses 66 in the form of elongate apertures or grooves along upper sides of the tip support body 14, and/or along the tapered second end 30, that are sized and shaped to receive portions of the flexible mirror strip 18. FIGS. 6-8 illustrate a different embodiment, in which the tip support body 14 may include one or more protrusions 70 (e.g., protruding ledges, sliding stops as illustrated in FIG. 6B) along upper sides of the tip support body 14 that are sized and shaped to constrain and/or otherwise couple to portions of the flexible mirror strip 18. FIGS. 9 and 10 illustrate yet a different embodiment, in which the tip support body 14 may include a first protrusion 74 (e.g., bump or knob) generally at or adjacent the first end 26 of the tip support body 14, and a second protrusion 78 (e.g., bump or knob) generally at or adjacent the second end 30 of the tip support body 14. Other embodiments include various other types, sizes, shapes, and arrangements of protrusions and/or recesses than that illustrated.

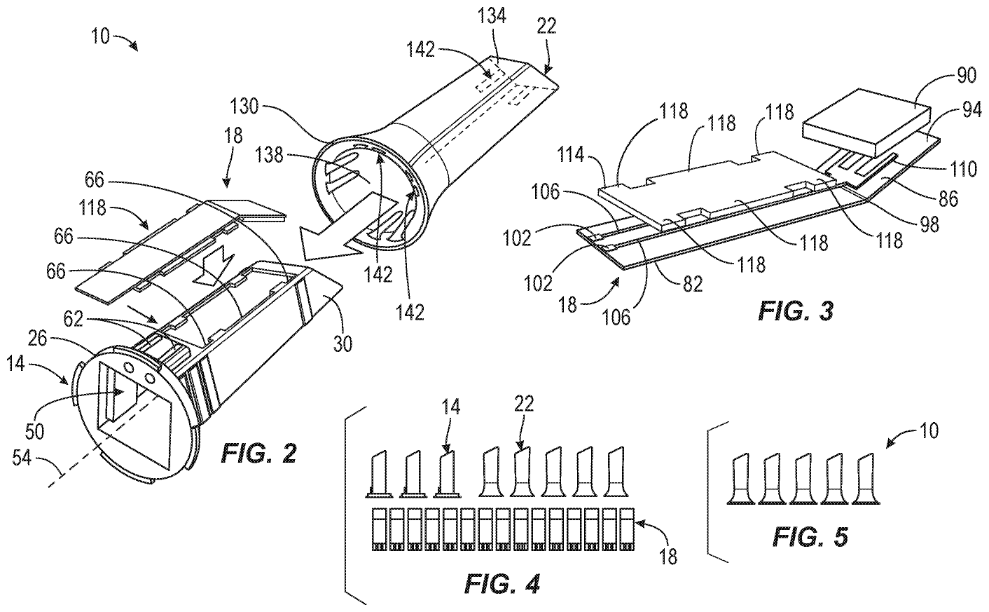

With reference to FIGS. 1-5, in the illustrated embodiment the flexible mirror strip 18 includes an elongate, flexible body having a first end 82, a second, opposite end 86, and a mirror 90 fixed (e.g., with adhesive 94 as illustrated in FIG. 3, for example, thermally conductive adhesive) to the second end 86. In other embodiments the mirror 90 is fixed, for example, with tape or another element. In the illustrated embodiment the flexible mirror strip 18 is an elongate, disposable, single-use strip. Thus, the mirror 90 is disposed of with the rest of the flexible mirror strip 18 with each use of the tip assembly 10. The flexible mirror strip 18 may be laminated for example with polyimide, although other embodiments include different materials. As illustrated in FIG. 3, the flexible mirror strip 18 may be bent at a bend or crease 98 adjacent the second end 86, such that the second end 86 is angled relative to the first end 82. In other embodiments the flexible mirror strip 18 may be straight, without a bend or crease 98. In some embodiments, the flexible mirror strip 18 is an elongate strip having a length as measured between the first and second ends 82, 86, and a width extending perpendicular to the length, where the width is smaller than the length, and where the width can be constant along the length of the flexible mirror strip.

With references to FIGS. 1 and 3, and as noted above, the tip assembly 10 includes a heating system that is used to remove condensation from the mirror 90. For example, the first end 82 of the flexible mirror strip 18 includes second electrical contacts 102 that physically engage the first electrical contacts 62 on the tip support body 14. As illustrated in FIG. 3, the flexible mirror strip 18 may include electrical connectors 106 (e.g., conductive wires or strips), and a resistor 110 (e.g., printed resistor) disposed beneath the mirror 90. The electrical connectors 106 extend from the second electrical contacts 102 to the resistor 110 to deliver current to the resistor 110 and heat the mirror 90. Thus, the resistor 110 is electrically coupled to the second electrical contacts 102. The flexible mirror strip 18 may further include a nonconductive element 114 that extends over the electrical connectors 106 between the first and second ends 82, 86 of the flexible mirror strip 18. Other methods for heating the mirror 90, for example, using a channel in the tip assembly 10 to provide heat from the tip support body 14, may be used.

Similar to the tip support body 14, the flexible mirror strip 18 may include at least one protrusion or recess which engages the at least one protrusion or recess of the tip support body 14 to releasably couple the flexible mirror strip 18 to the hollow tip support body 14. For example, and as illustrated in FIGS. 2 and 3 and 6C, the flexible mirror strip 18 may include multiple protrusions 118 (e.g., ribs or tabs) on the nonconductive element 114. As illustrated in FIGS. 9-11, in other embodiments the flexible mirror strip 18 may include a first recess 122 (e.g., aperture) generally at or adjacent the first end 82 of the flexible mirror strip 18, and a second recess 126 (e.g., aperture) generally at or adjacent the second end 86 of the flexible mirror strip 18. Other embodiments include various other types, sizes, shapes, and arrangements of protrusions and/or recesses than that illustrated. In some embodiments, the flexible mirror strip 18 does not include any protrusions or recesses for engagement with the tip support body 14. Rather, the flexible mirror strip 18 includes for example an elongate, solid strip of material. The flexible mirror strip 18 may be pressed down, and may snap underneath a protrusion or into a recess of the tip support body 14 or the tip sleeve 22. In yet other embodiments, the flexible mirror strip 18 may simply lie on top of the tip support body 14, and may be held in place on top of the tip support body 14 for example by friction when tip sleeve 22 slides over the combined flexible mirror strip 18 and tip support body 14.

With reference to FIGS. 1-5, the tip sleeve 22 is sized and shaped to slide over at least a portion of both the tip support body 14 and the flexible mirror strip 18. The tip sleeve 22 may be disinfected and/or sterilized for repeated use, or may be a single use device. For example, in some embodiments the tip sleeve 22 may be subjected to e.g. autoclave, UV light, or liquid disinfectant, to sterilize the tip sleeve 22. As illustrated in FIG. 2, the tip sleeve 22 has a first end 130 and a second, opposite end 134, and defines a hollow interior 138. In some embodiments, the tip sleeve 22 includes one or more internal features 142 (e.g., ribs, guides, channels, etc.) that facilitate and/or guide connection (e.g., snapping) of the tip sleeve 22 over the tip support body 14, and/or support the mirror 90 and the second end 86 of the flexible mirror strip 18. With reference to FIG. 1, the tip sleeve 22 further includes at least one aperture 146 at the second end 134 that aligns with the mirror 90. The aperture 146 is sized and shaped to receive light and allow the light to enter the tip assembly 10 and reach the mirror 90, where the light is reflected down the cavity 50. In the illustrated embodiment both the mirror 90 and the aperture 146 are rectangular, although in other embodiments the mirror 90 and aperture 146 may have different sizes and shapes than that illustrated.

With reference to FIGS. 2 and 3, in some embodiments a process for assembling the tip assembly 10 may include first aligning the flexible mirror strip 18 relative to the tip support body 14 such that the mirror 90 is aligned with the second end 30 of the tip support body 14 and the first electrical contacts 62 are aligned with the second electrical contacts 102. The flexible mirror strip 18 may then be pressed down onto the tip support body 14 until the protrusions 118 engage (e.g., snap) into the recesses 66. The tip sleeve 22 may then be slid over the combined tip support body 14 and flexible mirror strip 18, with the flange 38 and protrusions 42 still being disposed outside of the tip sleeve 22. In some embodiments, the flexible mirror strip 18 may be pressed first into the tip sleeve 22, and the combined tip sleeve 22 and flexible mirror strip 18 may then be slid over the tip support body 14. As illustrated in FIGS. 4 and 5, in some embodiments an array of tip support bodies 14, flexible mirror strips 18, and tip sleeves 22 may be provided prior to use (e.g., in a kit), ready for assembly. In yet other embodiments multiple tip assemblies 10 may be fully assembled prior to use.

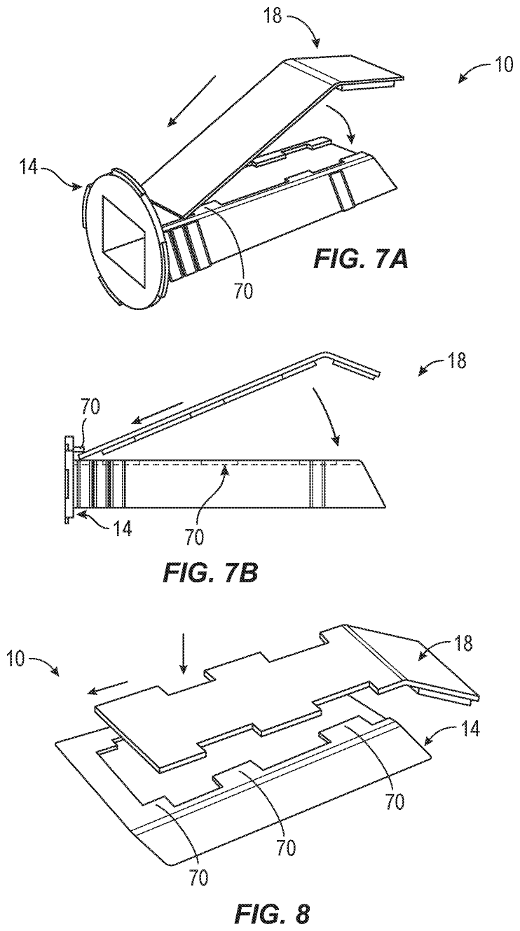

With reference to FIGS. 6 and 7, in yet other embodiments an assembly process may include aligning one side, or end, of the flexible mirror strip 18 such that one or more of the protrusions 118 (or the side or end itself of the flexible mirror strip 18) extends and slides underneath a protrusion 70 (e.g., protruding ledge as seen in FIG. 7B) on the tip support body 14. The flexible mirror strip 18 may then be pivoted down and, for example, snapped or otherwise held into place (e.g., frictionally).

With reference to FIG. 8, in yet other embodiments an assembly process may include pressing the flexible mirror strip 18 down vertically at first onto the tip support body 14 along a first direction, and then sliding the flexible mirror strip 18 in a second direction (e.g., perpendicularly) until protrusions 118 on the flexible mirror strip 18 extend under protrusions 70 on the tip support body 14, in a bayonet-like connection. In some embodiments, the flexible mirror strip 18 may not include any protrusions 118, but may still be pressed down for example and then slid under protrusions 70 on the tip support body 14 to lock the flexible mirror strip 18 in place.

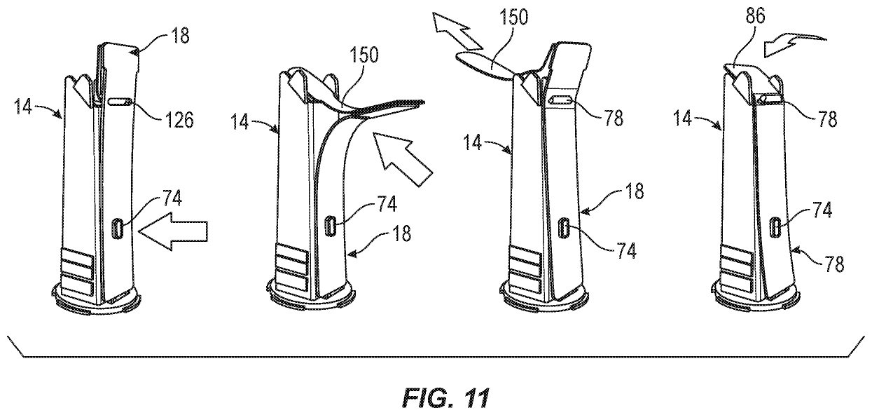

With reference to FIGS. 9 and 10, in some embodiments an assembly process may include removing a film or other protective cover layer 150 that extends over the mirror 90. Once the cover layer 150 is removed, the flexible mirror strip 18 may then be placed over the tip support body 14 such that the first recess 122 (e.g., aperture) snaps or otherwise extends over the first protrusion 74 (e.g., knob). The second recess 126 (e.g., aperture) may then be snapped or otherwise extended over the second protrusion 78 (e.g., knob). In yet other embodiments, and as illustrated in FIG. 11, the cover layer 150 may be removed after one or both of the first recess 122 and the second recess 126 have snapped over the first protrusion 74 and the second protrusion 78, respectively. As described above, in some embodiments the flexible mirror strip 18 may not include any recesses or protrusions. Thus, the flexible mirror strip 18 may be pressed down onto the tip support body 14 (for example snapped onto the tip support body 14 or sandwiched between the tip support body 14 and the tip sleeve 22 and held in place frictionally). The protective cover layer 150 may be removed before or after the flexible mirror strip 18 is snapped onto or otherwise positioned on top of the support body 14. In some embodiments, the protective cover layer 150 may be removed after the tip sleeve 22 has been slid over the combined flexible mirror strip 18 and tip support body 14. For example, once the tip sleeve 22 is slid over the combined flexible mirror strip and tip support body 14, the protective cover layer 150 may then be pulled out through the aperture 146 on the tip sleeve 22.

As illustrated in FIGS. 9-11, the second end 86 of the flexible mirror strip 18, which includes the mirror 90, may be bent and flex down over the tapered, second end 30 of the tip support body 14. In some embodiments, and as described above, the second end 30 may include a guide element (e.g., groove, rail, etc.) that helps to retain the bent second end 86. As illustrated in FIG. 10, once the flexible mirror strip 18 has been coupled to the tip support body 14, the tip sleeve 22 may then slide over the assembled combination.

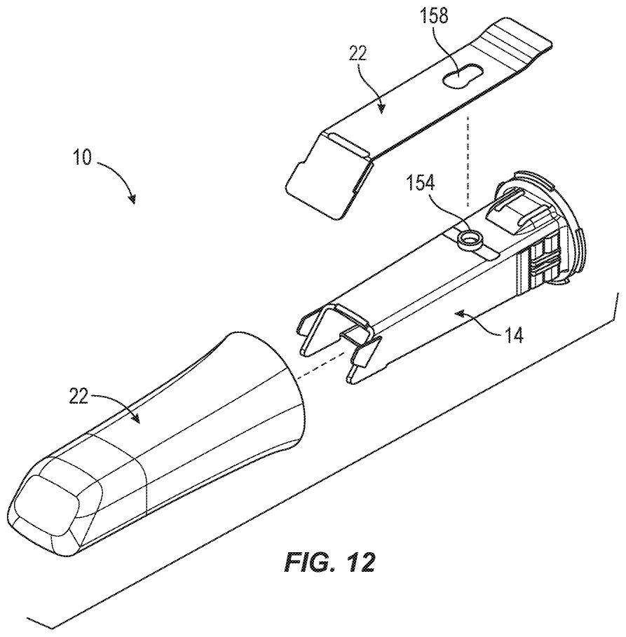

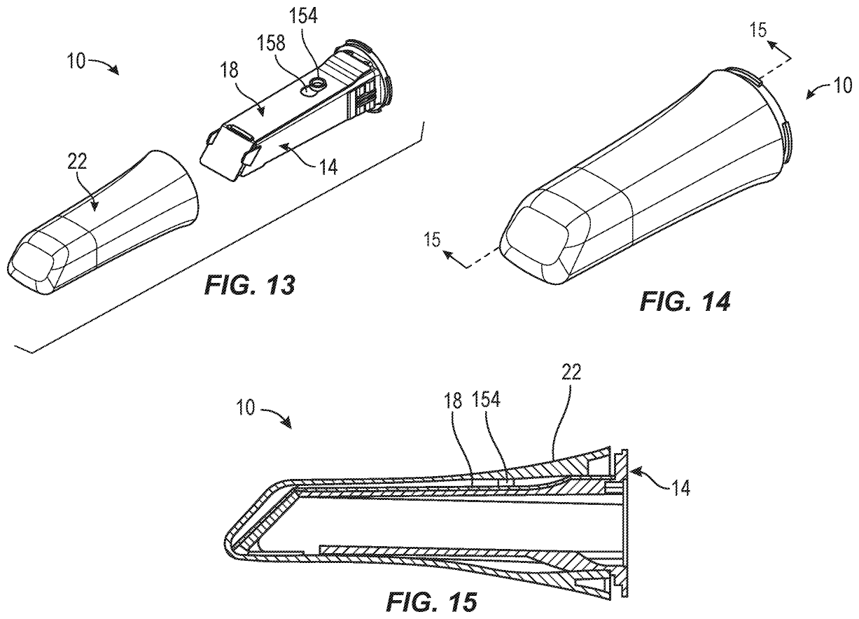

With reference to FIGS. 12-15, in some embodiments the tip support body 14 may include a protrusion (e.g., knob) 154 along an upper surface. The flexible mirror strip 18 includes a corresponding recess (e.g., aperture) 158 that fits over the protrusion 154. As illustrated in FIGS. 12 and 13, the recess 158 has regions of different size (e.g., diameter), such that when the recess 158 is placed over the protrusion 154 the flexible mirror strip 18 may be pushed or slid until the protrusion 154 is moved (e.g., snapped) into a different area of the recess 158 (e.g., having a smaller size), and the flexible mirror strip 18 is thus prevented or inhibited from being removed from the tip support body 14. As illustrated in FIGS. 14 and 15, when the tip sleeve 22 is coupled over the tip support body 14 and the flexible mirror strip 18, the protrusion 154 is concealed.

Once any of the tip assemblies described above have been assembled, the tip assembly may then be coupled (e.g., via a bayonet connection or other connection) to the rest of the intraoral scanner. For example, and as described above, in some embodiments the first end 26 of the tip support body 14 may include an outer (e.g., cylindrical) flange 38 and a series of ribs or protrusions 42 extending from the flange 38 that may be used to releasably couple and lock (e.g., via a bayonet connection) the tip support body 14 to the rest of the intraoral scanner. During use, the mirror 90 reflects light down the cavity 50 (e.g., to a camera in the intraoral scanner), and the mirror 90 is heated via the resistor 110 to prevent or inhibit build-up of condensation.

Once the tip assembly 10 has been used, the tip assembly 10 may be removed from the rest of the intraoral scanner, and the tip sleeve 22 may be pulled off and either discarded or autoclaved and/or sterilized. The flexible mirror strip 18 may then be removed (e.g., unsnapped) from the tip support body 14, and discarded. The tip support body 14 may then be autoclaved and/or sterilized, and prepared for re-use.

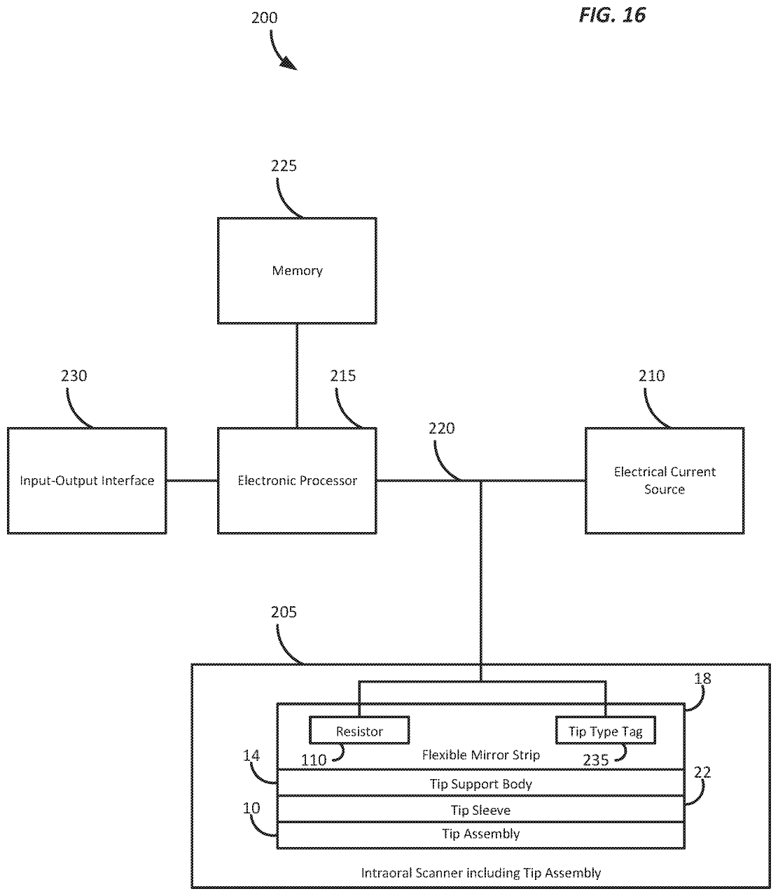

FIG. 16 illustrates an intraoral scanning system 200 according to one embodiment. The intraoral scanning system 200 includes an intraoral scanner 205 (illustrated in a block diagram format) that includes the tip assembly 10 (FIGS. 1 and 3). The intraoral scanner 205 is electrically connected to an electrical current source 210 and an electronic processor 215. For example, the first electrical contacts 62 (FIGS. 1 and 2) are connected to the electrical current source 210 and to the electronic processor 215 by electrical wiring 220 extending through the flange 38 (FIGS. 1 and 2) and out of the intraoral scanner 205. The electrical current source 210 provides an electrical current to the first electrical contacts 62, which in turn provides the electrical current to the resistor 110 via the second electrical contacts 102 (FIGS. 1 and 3).

The electronic processor 215 is electrically connected to the electrical current source 210 and the intraoral scanner 205. The electronic processor 215 is configured, in some embodiments, to control the electrical current source 210 to provide the current to the resistor 110.

Although FIG. 16 illustrates a system 200 in which the electrical current source 210, the electronic processor 215, a non-transitory, computer-readable memory 225, and input-output interface 230 are separate from the intraoral scanner 205, it should be understood that the components of the system 200 may all be wholly contained within the intraoral scanner 205. For example, the intraoral scanner 205 may be a handheld tool that contains the components of the system 200 within a handle of the handheld tool. In this example, the electrical current source 210 is, for example, a battery.

The electronic processor 215 is also configured to identify a tip assembly type of the tip assembly 10. In one embodiment, the tip assembly 10 is identified based upon a resistance of the resistor 110. For example, the resistor 110 may include a contact (for example, the second electrical contacts 102) that connects the resistor 110, via the first electrical contacts 62, to the electrical wiring 220 and the electronic processor 215. The electronic processor 215 determines the resistance of the resistor 110 and, based upon the determined resistance, identifies the tip assembly 10. The resistance may be compared to a look-up table stored in the memory 225 to identify the tip assembly 10. Based upon the comparison to the look-up table, the electronic processor 215 determines a type of the tip assembly 10. It is to be understood that other methods of determining the type of tip assembly 10 using the determined resistance of the resistor 110 may be used. For example, an analog-to-digital convertor may be used to determine the resistance of the resistor 110.

In some embodiments, the type of the tip assembly 10 is identified via a tag 235 stored, for example, in the flexible mirror strip 18. The tag 235 can also be stored in the tip support body 14 or the tip sleeve 22. The tag 235 includes an identifier of the tip assembly 10 and may be, for example, a radio frequency identification (“RFID”) tag or some other form of electronic identification tag. If the tag is stored in the flexible mirror strip 18, the tag 235 may be electrically connected via the first electrical contacts 62 to the electronic processor 215 and the electronic processor 215 is configured to detect the tip type of the tip assembly from the identification on the stored tag 235 via the electrical connection. If the tag 235 is, for example, an RFID tag, the electronic processor 215 may communicatively connect to the tag 235 via input-output interface 230, which may include a wireless transceiver used to wirelessly identify the RFID tag. The wireless transceiver detects the identification on the stored tag 235 by receiving returned identification data from the tag 235 in response to transmitting radio waves to the tag 235. The wireless transceiver then sends the detected identification to the electronic processor 215 to identify the tip assembly 10. It is to be understood that other methods of wirelessly detecting an identification that do not include using RFID may be used to identify the tip type.

While both the resistor 110 and the tip type tag 235 are illustrated in FIG. 16, in some embodiments, only one of the two elements is used to determine the tip type. For example, if the tip type tag 235 is included in the tip assembly 10, the electronic processor 215 is not configured to measure the resistance of the resistor 110 to determine the tip type. In contrast, if the resistance of the resistor 110 is used to determine the tip type, the tip type tag 235 is not included in the tip assembly 10.

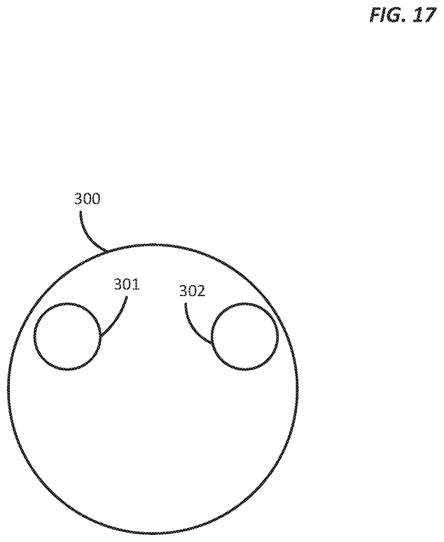

In other embodiments, the type of the tip assembly 10 is determined using one or more electrical contacts. FIG. 17 illustrates an example of a connector having two electrical contacts. In one embodiment, the flexible mirror strip 18 includes a number of studs or pins that, when the flexible mirror strip 18 is releasably coupled to the tip support body 14, connect with an electrical connection 300 located on the tip support body 14 via one or more connection points 301-302. Based upon the position and number of studs or tips contacting one or more of the connection points 301-302, the tip type is identified. This may be accomplished by transmitting a number of electrical signals to the electronic processor 215 via the electrical connection 300. The electronic processor 215 uses the electrical signals to determine the tip type. While the electrical connection 300 and the connection points 301-302 are illustrated as being circular, it is to be understand that the electrical connection 300 and the connection points 301-302 may be other shapes or sizes. In addition, the studs or pins may be located at various points on the flexible mirror strip 18 and not solely at the location described. Additionally, the studs or pins may not contain electrical leads, but may instead push or otherwise physically interact with the connection points 301-302, which in turn would identify the tip assembly 10 by electrically detecting which of the connection points 301-302 were physically interacted with by the different studs or pins. In one example, the connection points 301-302 are not located on the tip support body 14, but instead on the tip sleeve 22.

The tip type may indicate, for example, a shape and/or size of the mirror 90. In some embodiments, the tip type is used to control the intraoral scanner 205. In one example, the electronic processor 215 may alter operation of the intraoral scanner based upon the identified tip type, for example, by adjusting image processing to account for different sizes and types or mirror and to control the electrical current source 210 to adjust the amount of current delivered to the resistor 110 in order to heat the mirror 90.

The following examples illustrate example systems and methods described herein. Example 1: a tip assembly for an intraoral scanner, the tip assembly comprising a tip support body having a first end, a second, opposite end, and an outer surface located between the first end and the second end; a disposable, flexible mirror strip having a first end, a second, opposite end, and a mirror fixed to the second end; and a tip sleeve having a first end and a second, opposite end, wherein the tip sleeve is sized and shaped to slide over at least a portion of both the tip support body and the flexible mirror strip.

Example 2: the tip assembly of example 1, wherein the first end of the tip support body includes a first electrical contact along the outer surface, and wherein the first end of the flexible mirror strip includes a second electrical contact configured to physically engage the first electrical contact.

Example 3: the tip assembly of example 2, wherein the flexible mirror strip includes a resistor at the second end of the flexible mirror strip, and wherein the resistor is electrically coupled to the second electrical contact.

Example 4: the tip assembly of example 3, wherein the resistor is disposed at least partially beneath the mirror, and is configured to heat the mirror to remove condensation from the mirror.

Example 5: the tip assembly of any of examples 1-4, wherein the flexible mirror strip includes a nonconductive element disposed between the first end and the second end of the flexible mirror strip.

Example 6: the tip assembly of any of claims 1-5, wherein the outer surface of the tip support body defines at least one protrusion or recess, and wherein the flexible mirror strip defines at least one protrusion or recess configured to engage the at least one protrusion or recess of the tip support body to releasably couple the flexible mirror strip assembly to the tip support body, wherein the at least one protrusion or recess of the tip support body includes a plurality of recesses along the outer surface of the tip support body, and wherein the at least one protrusion or recess of the flexible mirror strip includes a plurality of protrusions configured to snap into the plurality of recesses to couple the flexible mirror strip onto the tip support body via frictional engagement.

Example 7: the tip assembly of any of examples 1-5, wherein the outer surface of the tip support body defines at least one protrusion or recess, and wherein the flexible mirror strip defines at least one protrusion or recess configured to engage the at least one protrusion or recess of the tip support body to releasably couple the flexible mirror strip assembly to the tip support body, wherein the at least one protrusion or recess of the tip support body includes a plurality of recesses along the outer surface of the tip support body, and wherein the at least one protrusion or recess of the flexible mirror strip includes a plurality of protrusions having shapes smaller than that of the recesses, such that the plurality of protrusions are configured to first pass through the recesses along a first direction, and the flexible mirror strip is then configured to slide in a second direction perpendicular to the first direction until the protrusions are disposed underneath the outer surface of the tip support body.

Example 8: the tip assembly of any of examples 1-5, wherein the outer surface of the tip support body defines at least one protrusion or recess, and wherein the flexible mirror strip defines at least one protrusion or recess configured to engage the at least one protrusion or recess of the tip support body to releasably couple the flexible mirror strip assembly to the tip support body, wherein the at least one protrusion or recess of the tip support body includes a protruding ledge, and wherein the first end of the flexible mirror strip is configured to slide under the protruding ledge to couple the flexible mirror strip to the tip support body.

Example 9: the tip assembly of any of examples 1-5, wherein the outer surface of the tip support body defines at least one protrusion or recess, and wherein the flexible mirror strip defines at least one protrusion or recess configured to engage the at least one protrusion or recess of the tip support body to releasably couple the flexible mirror strip assembly to the tip support body, wherein the at least one protrusion or recess of the tip support body includes a first protrusion adjacent the first end of the tip support body and a second protrusion adjacent the second end of the tip support body, wherein the at least one protrusion or recess of the flexible mirror strip includes a first recess adjacent the first end of the flexible mirror strip and a second recess adjacent the second end of the flexible mirror strip, wherein the first protrusion is configured to extend through the first recess and the second protrusion is configured to extend through the second recess.

Example 10: the tip assembly of any of examples 1-9, wherein the second end of the tip support body is tapered, and wherein the second end of the flexible mirror strip is configured to flex and bend over the tapered second end of the tip support body.

Example 11: the tip assembly of any of examples 1-10, wherein the first end of the tip support body includes a flange and a plurality of protrusions extending from the flange.

Example 12: the tip assembly of any of examples 1-9 and 11, wherein the second end of the tip support body includes two tapered arms separated by a gap.

Example 13: the tip assembly of any of examples 1-12, wherein the flexible mirror strip is an elongate strip having a length as measured between the first and second ends of the flexible mirror strip, and a width extending perpendicular to the length, wherein the width is smaller than the length, and wherein the width is constant along the length of the flexible mirror strip.

Example 14: the tip assembly of any of examples 1-13, wherein the flexible mirror strip is laminated with polyimide, and includes a printed resistor.

Example 15: the tip assembly of any of examples 1-14, wherein the first end of the tip sleeve is circular in cross-section.

Example 16: the tip assembly of any of examples 1-15, wherein the tip sleeve includes a plurality of internal features configured to guide the tip sleeve over the tip support body.

Example 17: the tip assembly of any of examples 1-16, wherein the second end of the tip sleeve includes an aperture sized and shaped to receive light when the tip sleeve is coupled to the tip support body and the flexible mirror strip.

Example 18: the tip assembly of any of examples 1-17, wherein the tip sleeve and the tip support body are both autoclavable.

Example 19: the tip assembly of any of examples 1-18, wherein the flexible mirror strip is coupled to the outer surface of the tip support body, and the tip sleeve is disposed over the flexible mirror strip.

Example 20: the tip assembly of any of examples 1-19, wherein the first end of the tip support body includes a flange and a plurality of protrusions extending from the flange, wherein the flange and protrusions are disposed outside of the tip sleeve.

Example 21: a disposable, flexible mirror strip for an intraoral scanner, the disposable, flexible mirror strip comprising an elongate, flexible body having a first end and a second, opposite end; and a mirror fixed to the second end.

Example 22: the disposable, flexible mirror strip of example 21, further comprising an electrical contact at the first end.

Example 23: the disposable, flexible mirror strip of example 22, further comprising a resistor disposed at least partially under the mirror, and an electrical connector extending from the electrical contact to the resistor, wherein the resistor is configured to heat the mirror, and wherein the electrical connector is configured to indicate a type of tip assembly being used.

Example 24: the disposable, flexible mirror strip of any of examples 21-23, further comprising a removable protective cover layer disposed over the mirror.

Example 25: a tip assembly for an intraoral scanner, the tip assembly comprising the disposable, flexible mirror strip of any of examples 21-24.

Example 26: an intraoral scanner comprising a tip assembly, an electrical current source, and an electronic processor configured to determine a tip type of the tip assembly.

Example 27: the intraoral scanner of example 26, wherein the tip assembly includes a flexible mirror strip, the flexible mirror strip comprising a resistor configured to heat the mirror.

Example 28: the intraoral scanner of example 27, wherein the electronic processor is further configured to control the electrical current source to provide current to the resistor to heat the mirror.

Example 29: the intraoral scanner of any of examples 27-28, wherein the electronic processor determines the tip type of the tip assembly by determining a resistance of the resistor.

Example 30: the intraoral scanner of any of examples 26-28, wherein the tip assembly includes a tip type tag, and wherein the electronic processor determines the tip type of the tip assembly based upon data received from the tip type tag.

Example 31: the intraoral scanner of example 30, wherein the electronic processor is electrically connected to the tip type tag.

Example 32: the intraoral scanner of example 30, wherein the electronic processor is wirelessly connected to the tip type tag.

Example 33: the intraoral scanner of any of examples 26-27 or 30-31, wherein the tip assembly includes a number of pins, and wherein the electronic processor determines the tip type of the tip assembly based on the number of pins.

Example 34: the intraoral scanner of any of examples 26-33, wherein the electronic processor is configured to alter a provided current to the intraoral scanner based upon the tip type.

Example 35: the intraoral scanner of any of examples 26-34, wherein the electronic processor is configured to alter operation of the intraoral scanner based upon the tip type.

Various features, aspects, and embodiments are set forth in the following claims.

What is claimed is:

1. A tip assembly for an intraoral scanner, the tip assembly comprising:
a tip support body having a first end, a second, opposite end, and an outer surface located between the first end and the second end;
a disposable, flexible mirror strip having a first end, a second, opposite end, and a mirror fixed to the second end; and
a tip sleeve having a first end and a second, opposite end, wherein the tip sleeve is sized and shaped to slide over at least a portion of both the tip support body and the flexible mirror strip.

2. The tip assembly of claim 1, wherein the first end of the tip support body includes a first electrical contact along the outer surface, and wherein the first end of the flexible mirror strip includes a second electrical contact configured to physically engage the first electrical contact.

3. The tip assembly of claim 2, wherein the flexible mirror strip includes a resistor at the second end of the flexible mirror strip, and wherein the resistor is electrically coupled to the second electrical contact.

4. The tip assembly of claim 3, wherein the resistor is disposed at least partially beneath the mirror, and is configured to heat the mirror to remove condensation from the mirror.

5. The tip assembly of claim 1, wherein the flexible mirror strip includes a nonconductive element disposed between the first end and the second end of the flexible mirror strip.

6. The tip assembly of claim 1, wherein the outer surface of the tip support body defines at least one protrusion or recess, and wherein the flexible mirror strip defines at least one protrusion or recess configured to engage the at least one protrusion or recess of the tip support body to releasably couple the flexible mirror strip assembly to the tip support body, wherein the at least one protrusion or recess of the tip support body includes a plurality of recesses along the outer surface of the tip support body, and wherein the at least one protrusion or recess of the flexible mirror strip includes a plurality of protrusions configured to snap into the plurality of recesses to couple the flexible mirror strip onto the tip support body via frictional engagement.

7. The tip assembly of claim 1, wherein the outer surface of the tip support body defines at least one protrusion or recess, and wherein the flexible mirror strip defines at least one protrusion or recess configured to engage the at least one protrusion or recess of the tip support body to releasably couple the flexible mirror strip assembly to the tip support body, wherein the at least one protrusion or recess of the tip support body includes a plurality of recesses along the outer surface of the tip support body, and wherein the at least one protrusion or recess of the flexible mirror strip includes a plurality of protrusions having shapes smaller than that of the recesses, such that the plurality of protrusions are configured to first pass through the recesses along a first direction, and the flexible mirror strip is then configured to slide in a second direction perpendicular to the first direction until the protrusions are disposed underneath the outer surface of the tip support body.

8. The tip assembly of claim 1, wherein the outer surface of the tip support body defines at least one protrusion or recess, and wherein the flexible mirror strip defines at least one protrusion or recess configured to engage the at least one protrusion or recess of the tip support body to releasably couple the flexible mirror strip assembly to the tip support body, wherein the at least one protrusion or recess of the tip support body includes a protruding ledge, and wherein the first end of the flexible mirror strip is configured to slide under the protruding ledge to couple the flexible mirror strip to the tip support body.

9. The tip assembly of claim 1, wherein the outer surface of the tip support body defines at least one protrusion or recess, and wherein the flexible mirror strip defines at least one protrusion or recess configured to engage the at least one protrusion or recess of the tip support body to releasably couple the flexible mirror strip assembly to the tip support body, wherein the at least one protrusion or recess of the tip support body includes a first protrusion adjacent the first end of the tip support body and a second protrusion adjacent the second end of the tip support body, wherein the at least one protrusion or recess of the flexible mirror strip includes a first recess adjacent the first end of the flexible mirror strip and a second recess adjacent the second end of the flexible mirror strip, wherein the first protrusion is configured to extend through the first recess and the second protrusion is configured to extend through the second recess.

10. The tip assembly of claim 1, wherein the second end of the tip support body is tapered, and wherein the second end of the flexible mirror strip is configured to flex and bend over the tapered second end of the tip support body.

11. The tip assembly of claim 1, wherein the first end of the tip support body includes a flange and a plurality of protrusions extending from the flange.

12. The tip assembly of claim 1, wherein the second end of the tip support body includes two tapered arms separated by a gap.

13. The tip assembly of claim 1, wherein the flexible mirror strip is an elongate strip having a length as measured between the first and second ends of the flexible mirror strip, and a width extending perpendicular to the length, wherein the width is smaller than the length, and wherein the width is constant along the length of the flexible mirror strip.

14. The tip assembly of claim 1, wherein the flexible mirror strip is laminated with polyimide, and includes a printed resistor.

15. The tip assembly of claim 1, wherein the first end of the tip sleeve is circular in cross-section.

16. The tip assembly of claim 1, wherein the tip sleeve includes a plurality of internal features configured to guide the tip sleeve over the tip support body.

17. The tip assembly of claim 1, wherein the second end of the tip sleeve includes an aperture sized and shaped to receive light when the tip sleeve is coupled to the tip support body and the flexible mirror strip.

18. The tip assembly of claim 1, wherein the tip sleeve and the tip support body are both autoclavable.

19. The tip assembly of claim 1, wherein the flexible mirror strip is coupled to the outer surface of the tip support body, and the tip sleeve is disposed over the flexible mirror strip.

20. The tip assembly of claim 19, wherein the first end of the tip support body includes a flange and a plurality of protrusions extending from the flange, wherein the flange and protrusions are disposed outside of the tip sleeve.

21. A disposable, flexible mirror strip for an intraoral scanner, the disposable, flexible mirror strip comprising:
an elongate, flexible body having a first end and a second, opposite end; and
a mirror fixed to the second end.

22. The disposable, flexible mirror strip of claim 21, further comprising an electrical contact at the first end.

23. The disposable, flexible mirror strip of claim 22, further comprising a resistor disposed at least partially under the mirror, and an electrical connector extending from the electrical contact to the resistor, wherein the resistor is configured to heat the mirror, and wherein the electrical connector is configured to indicate a type of tip assembly being used.

24. The disposable, flexible mirror strip of claim 21, further comprising a removable protective cover layer disposed over the mirror.

25. A tip assembly for an intraoral scanner, the tip assembly comprising the disposable, flexible mirror strip of claim 21.

26. An intraoral scanner comprising:
a tip assembly,
an electrical current source, and
an electronic processor configured to determine a tip type of the tip assembly.

27. The intraoral scanner of claim 26, wherein the tip assembly includes a flexible mirror strip, the flexible mirror strip comprising a resistor configured to heat the mirror.

28. The intraoral scanner of claim 27, wherein the electronic processor is further configured to control the electrical current source to provide current to the resistor to heat the mirror.

29. The intraoral scanner of claim 27, wherein the electronic processor determines the tip type of the tip assembly by determining a resistance of the resistor.

30. The intraoral scanner of claim 26, wherein the tip assembly includes a tip type tag, and wherein the electronic processor determines the tip type of the tip assembly based upon data received from the tip type tag.

31. The intraoral scanner of claim 30, wherein the electronic processor is electrically connected to the tip type tag.

32. The intraoral scanner of claim 30, wherein the electronic processor is wirelessly connected to the tip type tag.

33. The intraoral scanner of claim 26, wherein the tip assembly includes a number of pins, and wherein the electronic processor determines the tip type of the tip assembly based on the number of pins.

34. The intraoral scanner of claim 26, wherein the electronic processor is configured to alter a provided current to the intraoral scanner based upon the tip type.

35. The intraoral scanner of claim 26, wherein the electronic processor is configured to alter operation of the intraoral scanner based upon the tip type.
